(19) United States
(12) Patent Application Publication
(10) Pub. No.: US 2010/0099953 A1
(43) Pub. Date: Apr. 22, 2010

(54) MEDICAL DEVICE, MEDICAL EQUIPMENT AND ENDOSCOPE

(76) Inventors: Akira SUZUKI, Uenohara-shi (JP); Fumiyuki ONODA, Tama-shi (JP); Katsuyuki SAITO, Sagamihara-shi (JP); Seiji YAMAGUCHI, Hachioji-shi (JP); Kiyoshi MIYAKE, Niiza-shi (JP); Hitoshi SHIRAI, Sagamihara-shi (JP)

Correspondence Address: OSTROLENK FABER GERB & SOFFEN, 1180 AVENUE OF THE AMERICAS, NEW YORK, NY, 100368403, US

(21) Appl. No.: 12/644,198

(22) Filed: Dec. 22, 2009

Related U.S. Application Data
(63) Continuation of application No. PCT/JP2007/062713, filed on Jun. 25, 2007.

(51) Int. Cl. A61B 1/00

(52) U.S. Cl. 600/153

(57) ABSTRACT

A medical device includes a communication member including a first opening portion, a second opening portion, and a communication conduit for communication between the first opening portion and the second opening portion, a closing portion capable of liquid-tightly closing the communication conduit, and a maintaining member capable of maintaining the closing portion in a closed state.

### CROSS REFERENCE TO RELATED APPLICATIONS

This is a Continuation Application of PCT Application No. PCT/JP2007/062713, filed Jun. 25, 2007, which was published under PCT Article 21(2) in Japanese.

### BACKGROUND OF THE INVENTION

1. Field of the Invention

The present invention relates to a medical device, medical equipment and an endoscope for disposable use, which are discarded after single-time use without being reused.

2. Description of the Related Art

Conventionally, there is known a medical device for disposable use, which is discarded after single-time use without being reused. An example of such a medical device for disposable use is an endoscope system which is disclosed in Jpn. Pat. Appln. KOKAI Publication No. 2007-050108 (patent document 1). An endoscope for disposable use (hereinafter referred to as “disposable endoscope”) is provided with a memory which stores data indicating that the disposable endoscope has been used. The endoscope system is configured such that when the disposable endoscope is connected to a processor, the processor confirms the content of the memory, thereby detecting whether the disposable endoscope is before use or after use. The data relating to the use condition is read out of the memory, and it is determined whether the disposable endoscope has already been used. If it is determined that the disposable endoscope has already been used, the output of a video signal is stopped, and no video signal is input to a monitor. In addition, the monitor displays that the disposable endoscope has already been used. Thereby, the already used disposable endoscope is prevented from being erroneously reused.

### BRIEF SUMMARY OF THE INVENTION

According to a first aspect of the present invention, a medical device comprising: a communication member including a first opening portion, a second opening portion, and a communication conduit for communication between the first opening portion and the second opening portion; a closing portion capable of liquid-tightly closing the communication conduit; and a maintaining member capable of maintaining the closing portion in a closed state.

Preferably, a deformation portion provided on the communication conduit as the closing portion; and an input section which deforms the deformation portion.

Preferably, a first holding portion and a second holding portion which are provided on both sides of the deformation portion on the communication conduit; and a rotation section enabling relative rotation between the first holding portion and the second holding portion, with the deformation portion being interposed.

Preferably, a medical device body through which the communication conduit is passed; and an operation section which operates the input section from outside of the medical device body.

Preferably, the deformation portion is closed when the medical device is bent over a predetermined diameter or more; and the maintaining member maintains the state in which the medical device is bent over the predetermined diameter or more.

Preferably, a third opening portion and a fourth opening portion which are formed, with a gap portion interposed, between the first opening portion and the second opening portion which are provided on the communication conduit; the closing portion which is slidably provided at the gap portion and includes an opening window which permits communication between the third opening portion and the fourth opening portion, and a closing portion which enables closing between the third opening portion and the fourth opening portion; and the maintaining member capable of fixing the closing member in a state in which the third opening portion and the fourth opening portion are closed by the closing portion.

Preferably, an attachment portion which liquid-tightly attaches the closing portion to the first opening portion or the second opening portion; and an urging member for urging the closing portion in a closed state.

Preferably, the attachment portion which liquid-tightly attaches the closing portion to the first opening portion or the second opening portion; and an urging member for urging the closing portion in a closed state.

According to second aspect of the present invention, the medical device of claim 1; an external device to which the medical device is connected; and a communication maintaining member, provided on the external device, for preventing the closing portion from closing the communication conduit while the medical device is being used.

According to third aspect of the present invention, an endoscope including the medical device of claim 1, the endoscope including at least one of an insertion section and a universal cord, the closing portion being provided at least one of the insertion section and the universal cord.

### BRIEF DESCRIPTION OF THE SEVERAL VIEWS OF THE DRAWING

FIG. 1 is a schematic view of the entire structure of an endoscope, showing a communication conduit of a disposable endoscope according to a first embodiment of the present invention;

FIG. 2 is a longitudinal cross-sectional view of a main part of the entire endoscope, showing the state in which closing means of the communication conduit of the disposable endoscope of the first embodiment is held in an open position;

FIG. 3 is a longitudinal cross-sectional view of a main part of the entire endoscope, showing the state in which the closing means of the communication conduit of the disposable endoscope of the first embodiment is held in a closed position;

FIG. 4 is a longitudinal cross-sectional view of a main part of an entire endoscope, showing the state in which closing means of a communication conduit of a disposable endoscope according to a second embodiment of the invention is held in an open position;

FIG. 5 is a longitudinal cross-sectional view of a main part of the entire endoscope, showing the state in which the closing means of the communication conduit of the disposable endoscope according to the second embodiment is held in a closed position;

FIG. 6 is a longitudinal cross-sectional view of a main part, showing the state in which closing means of a communication conduit of a disposable endoscope according to a third embodiment of the invention is held in an open position;

FIG. 7 is a longitudinal cross-sectional view of a main part, showing the state in which the closing means of the communication conduit of the disposable endoscope according to the third embodiment is held in a closed position;

FIG. 8 is a perspective view of an entire endoscope, showing the state in which a disposable endoscope according to a fourth embodiment of the invention is held in a use state;

FIG. 9 is a longitudinal cross-sectional view of a main part, showing the state in which closing means of a flexible tube in the disposable endoscope according to the fourth embodiment is held in an open position;

FIG. 10 is a longitudinal cross-sectional view of a main part, showing the state in which one projection portion of the closing means of the flexible tube of the disposable endoscope according to the fourth embodiment is held in an open position;

FIG. 11 is a longitudinal cross-sectional view of a main part, showing the state in which one projection portion of the closing means of the flexible tube of the disposable endoscope according to the fourth embodiment is moved to a closed position;

FIG. 12 is a perspective view of the entire endoscope, showing the state in which the disposable endoscope according to the fourth embodiment is wound and discarded;

FIG. 13 is a perspective view showing a refuse bag attached to a drape placed over a cart which carries an endoscope system;

FIG. 14 is a plan view of a refuse-bag-contained package;

FIG. 15 is a perspective view showing the state in which a used endoscope is stored in a refuse bag;

FIG. 16 is a longitudinal cross-sectional view of a main part, showing the state in which closing means of a communication conduit of a disposable endoscope according to a fifth embodiment of the invention is held in an open position;

FIG. 17 is a longitudinal cross-sectional view of a main part, showing the state in which the closing means of the communication conduit of the disposable endoscope according to the fifth embodiment is held in a closed position;

FIG. 18 is a perspective view of a main part, showing the state in which closing means of a communication conduit of a disposable endoscope according to a sixth embodiment of the invention is held in a closed position;

FIG. 19 is a longitudinal cross-sectional view of a main part, showing the state in which the closing means of the communication conduit of the disposable endoscope according to the sixth embodiment is held in the closed position;

FIG. 20 is a longitudinal cross-sectional view of a main part, showing the state in which closing means of a communication conduit of a disposable endoscope according to a seventh embodiment of the invention is held in an open position;

FIG. 21 is a longitudinal cross-sectional view of a main part, showing the state in which the closing means of the communication conduit of the disposable endoscope according to the seventh embodiment is held in the open position;

FIG. 22 is a longitudinal cross-sectional view of a main part, showing the state in which the closing means of the communication conduit of the disposable endoscope according to the seventh embodiment is moved to a closed position;

FIG. 23 is a longitudinal cross-sectional view of a main part, showing the state in which closing means of a communication conduit of a disposable endoscope according to an eighth embodiment of the invention is held in an open position;

FIG. 24 is a longitudinal cross-sectional view of a main part, showing the state in which the closing means of the communication conduit of the disposable endoscope according to the eighth embodiment is moved to a closed position;

FIG. 25 is a longitudinal cross-sectional view of a main part, showing the state in which closing means of a communication conduit of a disposable endoscope according to a ninth embodiment of the invention is held in an open position;

FIG. 26 is a longitudinal cross-sectional view of a main part, showing the state in which the closing means of the communication conduit of the disposable endoscope according to the ninth embodiment is moved to a closed position;

FIG. 27 is a longitudinal cross-sectional view of a main part, showing the state in which closing means of a communication conduit of a disposable endoscope according to a tenth embodiment of the invention is held in a closed position;

FIG. 28 is a longitudinal cross-sectional view of a main part, showing a connector receiving portion on a medical equipment side, to which the disposable endoscope of the tenth embodiment is connected; and

FIG. 29 is a longitudinal cross-sectional view of a main part, showing the state in which the closing means of the communication conduit of the disposable endoscope according to the tenth embodiment is held in an open position.

### DETAILED DESCRIPTION OF THE INVENTION

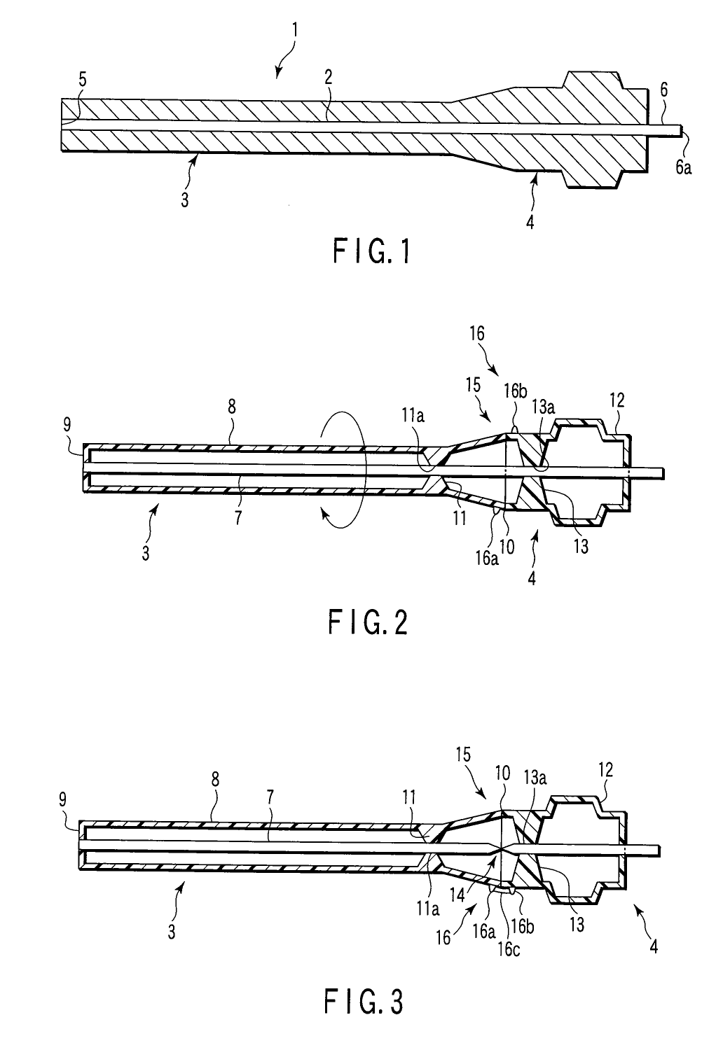

A first embodiment of the present invention will now be described with reference to FIG. 1 to FIG. 3. FIG. 1 schematically shows the structure of a disposable endoscope 1 which is a disposable medical device according to the first embodiment. An internal conduit (communication means) 2, such as a suction conduit (communication conduit), as well as an illumination optical system (not shown) and an observation optical system (not shown), is provided in the disposable endoscope 1.

The endoscope 1 includes an elongated insertion section 3 and a proximal-side operation section 4 which is coupled to a proximal end portion of the insertion section 3. One end portion of the conduit 2 communicates with a distal-end opening portion (first opening portion) 5 which is provided at a distal end portion of the insertion section 3 of the endoscope 1. The other end portion of the conduit 2 communicates with an opening end (second opening portion) 6a of a coupling end portion 6 which is provided on the operation section 4 of the endoscope 1. The coupling end portion 6 may be configured to serve also as a therapeutic device insertion hole portion, for instance.

As shown in FIG. 2, the conduit 2 of the disposable endoscope 1 is generally formed of a flexible tube 7. The body of the insertion section 3 of the disposable endoscope 1 is formed of, for example, a flexible tube body 8. A distal-end rigid portion 9 is provided on a distal end portion of the tube body 8. A distal end portion of the tube 7 is fixed to the distal-end rigid portion 9.

A coupling part between a proximal end portion of the tube body 8 and the operation section 4 is provided with a rotation section 10 which rotatably couples the proximal end portion of the tube body 8 and the operation section 4 such that the proximal end portion of the tube body 8 and the operation section 4 are rotatable about a center axis of the tube body 8. In addition, a first holding portion 11, which holds the tube 7, is projectingly provided on a proximal-end-side inner peripheral surface of the tube body 8. A tube insertion hole 11a is formed at a center axis portion of the first holding portion 11. The tube 7 is fixed to the first holding portion 11 in the state in which the tube 7 is passed through the tube insertion hole 11a of the first holding portion 11.

A second holding portion 13, which holds the tube 7, is projectingly provided on a casing 12 of the operation section 4 in the vicinity of the rotation section 10. A tube insertion hole 13a is formed at a center axis portion of the second holding portion 13. The tube 7 is fixed to the second holding portion 13 in the state in which the tube 7 is passed through the tube insertion hole 13a of the second holding portion 13.

At a time of normal use, the disposable endoscope 1 of the present embodiment is held by a rotation prevention stopper (not shown) at a fixed position that is a non-rotatable position where the rotation section 10 is not rotated, as shown in FIG. 2. In this state, the tube 7 is held in an open state.

After use, the disposable endoscope 1 is rotated in the state in which the proximal end portion of the tube body 8 and the operation section 4 are twisted about the center axis of the tube body 8 via the rotation section 10. By this rotation operation, as shown in FIG. 3, a deformation portion 14, which is twisted and deformed in a closed state, is formed at an intermediate portion of the tube 7 between the first holding portion 11 and the second holding portion 13. At this time, the tube 7 is rotated over a rotation angle of, e.g. 180° or more, and thereby the internal conduit 2 of the tube 7 is liquid-tightly closed. In this manner, in the present embodiment, the first holding portion 11, second holding portion 13 and rotation section 10 constitute closing means 15 which can liquid-tightly close the internal conduit 2 of the tube 7 by the rotation of the rotation section 10.

Furthermore, the disposable endoscope 1 of the present embodiment is provided with maintaining means 16 which can keep the internal conduit 2 of the tube 7 in the closed state. The maintaining means 16 includes a first engaging projection portion 16a which is projectingly provided on an outer peripheral surface of the proximal end portion of the tube body 8, a second engaging projection portion 16b which is projectingly provided on an outer peripheral surface of a distal end portion of the operation section 4, and an engaging member 16c. The first engaging projection portion 16a and second engaging projection portion 16b are set in such a positional relationship that the first engaging projection portion 16a and second engaging projection portion 16b are spaced apart and opposed to each other when the rotation section 10 has been rotated from the fixed position to the closed position at which the internal conduit 2 of the tube 7 is liquid-tightly closed. The engaging member 16c is formed of a hook member having one end portion coupled to either the first engaging projection portion 16a or the second engaging projection portion 16b, for example, the second engaging projection portion 16b. When the rotation section 10 of the disposable endoscope 1 has been rotated to the closed position, the hook member of the engaging member 16c is engaged with the first engaging projection portion 16a, and the internal conduit 2 of the tube 7 is kept in the state in which the internal conduit 2 is closed by the closing means 15.

In the meantime, the maintaining means 16 is not limited to the above-described structure. For example, the rotation section 10 between the proximal end portion of the tube body 8 and the operation section 4 may be formed of a screw-coupling portion. In this case, screwing is effected up to the terminal end part of the screw-coupling portion at a time point when the rotation section 10 of the disposable endoscope 1 has been rotated to the closed position. Thereby, in this structure, the internal conduit 2 of the tube 7 is maintained in the state in which the internal conduit 2 is closed by the closing means 15.

Next, the operation of the above-described structure is described. At a time of normal use, the disposable endoscope 1 of the present embodiment is held by the rotation prevention stopper (not shown) at the fixed position that is the non-rotatable position where the rotation section 10 is not rotated, as shown in FIG. 2. In this state, the tube 7 is held in the open state.

After use, the disposable endoscope 1 is rotated in the state in which the proximal end portion of the tube body 8 and the operation section 4 are twisted about the center axis of the tube body 8 via the rotation section 10. By this rotation operation, as shown in FIG. 3, the intermediate portion of the tube 7 between the first holding portion 11 and the second holding portion 13 is twisted, and the deformation portion 14 is formed. At this time, the tube 7 is rotated over the rotation angle of, e.g. 180° or more, and thereby the internal conduit 2 of the tube 7 is liquid-tightly closed.

Subsequently, when the rotation section 10 of the disposable endoscope 1 has been rotated to the closed position, the hook member of the engaging member 16c is engaged with the first engaging projection portion 16a. Thereby, the rotation section 10 of the disposable endoscope 1 is fixed in the closed position. As a result, the internal conduit 2 of the tube 7 is kept in the closed state.

The following advantageous effects can be obtained by the above-described structure. Specifically, in the disposable endoscope 1 of the present embodiment, after use for, e.g. endoscopy, the proximal end portion of the tube body 8 and the operation section 4 are rotated via the rotation section 10 in the twisted state about the center axis of the tube body 8. Thereby, the intermediate part between the first holding portion 11 and second holding portion 13 is twisted and the deformation portion 14 is formed. Thus, the internal conduit 2 of the tube 7 is liquid-lightly closed. Thereby, the liquid, such as a body liquid, which has flown in the tube 7, can be prevented from flowing out of the internal conduit 2 of the tube 7. As a result, the liquid, such as a body liquid, which stays in the internal conduit 2 of the tube 7 of the disposable endoscope 1, which has already been used, does not leak to the outside from the internal conduit 2 of the tube 7. Therefore, after the disposable endoscope 1 has been used for an infected patient, it is possible to reduce occasions where the source of infection spreads.

In the present embodiment, there is disclosed the structure wherein the closing means 15, which can liquid-tightly close the internal conduit 2 of the tube 7, is formed at the coupling part between the proximal end portion of the tube body 8 and the operation section 4. In addition to this structure, another closing means 15 of the same structure may be formed at the distal end portion of the tube body 8. In this case, both end portions of the tube 7 of the disposable endoscope 1, which has already been used, can be closed. Therefore, advantageously, it becomes possible to more surely prevent the liquid, such as a body liquid, which stays in the internal conduit 2 of the tube 7, from leaking to the outside from the internal conduit 2 of the tube 7.

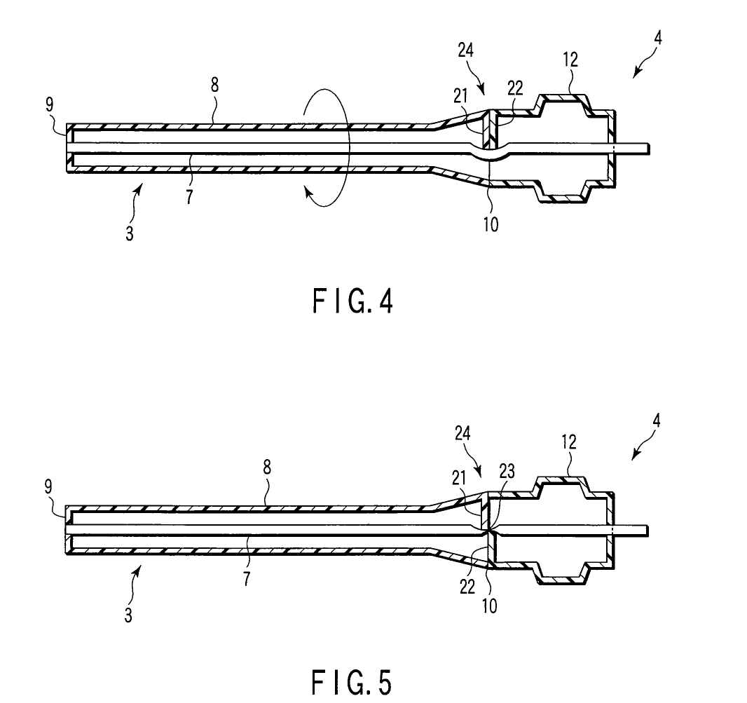

FIG. 4 and FIG. 5 show a second embodiment of the present invention. In the present embodiment, the structure of the closing means 15 of the disposable endoscope 1 of the first embodiment (see FIG. 1 to FIG. 3) is altered as described below. Except for this altered part, the structure of the second embodiment is the same as the structure of the disposable endoscope 1 of the first embodiment. The parts common to those of the disposable endoscope 1 of the first embodiment are denoted by like reference numerals, and a description thereof is omitted here.

Specifically, in the disposable endoscope 1 of the present embodiment, a first bent portion (first holding portion) 21, which is bent substantially at right angles toward the inside of the tube body 8, is formed at the position of the rotation section 10 on the proximal end side of the tube body 8. The first bent portion 21 is formed at an arbitrary angle of less than 180° along the circumferential direction of the inner peripheral surface of the tube body 8. The distal end of the first bent portion 21 extends to a position substantially corresponding to the center axis of the tube body 8 or to a position slightly beyond the center axis of the tube body 8. The tube 7 is fixed at the distal end position of the first bent portion 21.

A second bent portion 22, which has a shape corresponding to the first bent portion 21, is formed on the casing 12 of the operation section 4 at the position of the rotation section 10. The distal end of the second bent portion 22 extends to a position substantially corresponding to the center axis of the tube body 8 or to a position slightly beyond the center axis of the tube body 8. The tube 7 may be fixed at the distal end position of the second bent portion 22.

At a time of normal use, the disposable endoscope 1 of the present embodiment is held by a rotation prevention stopper (not shown) at a fixed position that is a non-rotatable position where the rotation section 10 is not rotated, as shown in FIG. 4. In this state, the first bent portion 21 and second bent portion 22 are held in the state in which the first bent portion 21 and second bent portion 22 overlap at substantially the same position in the rotational direction of the rotation section 10. At this time, the tube 7 is held in an open state.

After use, the disposable endoscope 1 is rotated in the state in which the proximal end portion of the tube body 8 and the operation section 4 are twisted about the center axis of the tube body 8 via the rotation section 10. By this rotation operation, as shown in FIG. 5, a deformation portion 23, which is twisted and deformed in a closed state, is formed at an intermediate portion of the tube 7 between the first bent portion 21 and second bent portion 22. At this time, the tube 7 is rotated over a rotation angle of, e.g. 180° or more, and thereby the internal conduit 2 of the tube 7 is liquid-tightly closed. In this manner, in the present embodiment, the first bent portion 21, second bent portion 22 and rotation section 10 constitute closing means 24 which can liquid-tightly close the internal conduit 2 of the tube 7 by the rotation of the rotation section 10.

Next, the operation of the above-described structure is described. At a time of normal use, the disposable endoscope 1 of the present embodiment is held by the rotation prevention stopper (not shown) at the fixed position that is the non-rotatable position where the rotation section 10 is not rotated, as shown in FIG. 4. In this state, the tube 7 is held in the open state.

After use, the disposable endoscope 1 is rotated in the state in which the proximal end portion of the tube body 8 and the operation section 4 are twisted about the center axis of the tube body 8 via the rotation section 10. By this rotation operation, as shown in FIG. 5, the intermediate portion of the tube 7 between the first bent portion 21 and the second bent portion 22 is twisted, and the deformation portion 23 is formed. At this time, the tube 7 is rotated over the rotation angle of, e.g. 180° or more, and thereby the internal conduit 2 of the tube 7 is liquid-tightly closed.

Subsequently, when the rotation section 10 of the disposable endoscope 1 has been rotated to the closed position, the rotation of the disposable endoscope 1 is fixed by the closing means 15 having the same structure as in the disposable endoscope 1 of the first embodiment. As a result, the internal conduit 2 of the tube 7 is kept in the closed state.

The following advantageous effects can be obtained by the above-described structure. Specifically, in the disposable endoscope 1 of the present embodiment, after use for, e.g. endoscopy, the proximal end portion of the tube body 8 and the operation section 4 are rotated via the rotation section 10 in the twisted state about the center axis of the tube body 8. Thereby, the intermediate part between the first bent portion 21 and second bent portion 22 is twisted and the deformation portion 23 is formed. Thus, the internal conduit 2 of the tube 7 is liquid-lightly closed. Thereby, the liquid, such as a body liquid, which has flown in the tube 7, can be prevented from flowing out of the internal conduit 2 of the tube 7. As a result, the liquid, such as a body liquid, which stays in the internal conduit 2 of the tube 7 of the disposable endoscope 1, which has already been used, does not leak to the outside from the internal conduit 2 of the tube 7. Therefore, after the disposable endoscope 1 has been used for an infected patient, it is possible to reduce occasions where the source of infection spreads.

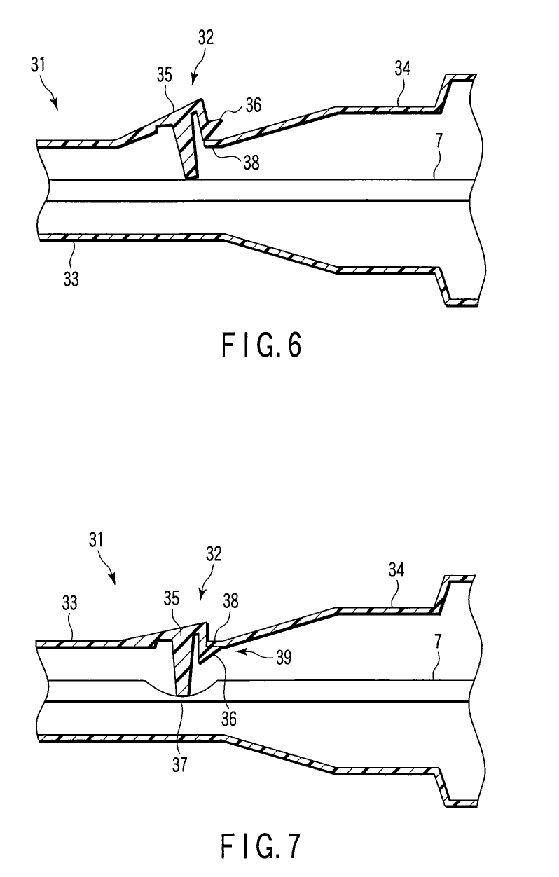

FIG. 6 and FIG. 7 show a third embodiment of the present invention. The disposable endoscope 1 of the first embodiment (see FIG. 1 to FIG. 3) is configured to have such an input section that the proximal end portion of the tube body 8 and the operation section 4 are rotated via the rotation section 10 in the twisted state about the center axis of the tube body 8, thereby twisting the tube 7 and closing the tube 7. In a disposable endoscope 31 of the present embodiment, the structure of an input section 32 at the time of closing the tube 7 is altered from the first embodiment, as described below.

Specifically, in the disposable endoscope 31 of the present embodiment, a coupling part between an elongated insertion section 33 and a proximal-side operation section 34 is provided with a push button 35 and a hook-shaped engaging claw 36. The push button 35 is supported at a proximal end portion of the insertion section 33 so as to be deformable from a non-pushed position shown in FIG. 6 to a pushed position shown in FIG. 7.

In addition, in the non-pushed position shown in FIG. 6, the push button 35 is held in the state of non-contact with the tube 7. In this state, the tube 7 is held in an open state. In the case where the push button 35 is pushed to the pushed position shown in FIG. 7, an inner end portion of the push button 35 pushes the tube 7, and collapses the tube 7. At this time, a deformation portion 37, which is collapsed by the push button 35 and deformed in the closed state, is formed on the tube 7.

A stopper 38, which is engaged with the engaging claw 36, is provided on a distal-end-side edge portion of the operation section 34. In the case in which the push button 35 is held in the non-pushed position shown in FIG. 6, the engaging claw 36 is held in a non-engagement position spaced apart from the stopper 38. In the case where the push button 35 is pushed to the pushed position shown in FIG. 7, the engaging claw 36 is engaged with the stopper 38. The engagement part between the engaging claw 36 and stopper 38 constitutes maintaining means 39 which can maintain the internal conduit 2 of the tube 7 in the closed state. The engaging claw 36 is configured such that the engaging claw 36, if once engaged with the stopper 38, cannot be disengaged.

Next, the operation of the above-described structure is described. At a time of normal use of the disposable endoscope 31 of the present embodiment, the push button 35 of the input section 32 is held in the non-pushed position shown in FIG. 6. In this state, the tube 7 is held in the open state.

After use of the disposable endoscope 31, the push button 35 of the input section 32 is pushed. At this time, in the case where the push button 35 has been pushed to the pushed position shown in FIG. 7, the inner end portion of the push button 35 pushes the tube 7, and the tube 7 is collapsed. Thereby, the tube 7 is collapsed by the push button 35, the deformation portion 37 is formed, and the internal conduit 2 of the tube 7 is liquid-tightly closed.

At the same time, the engaging claw 36 of the disposable endoscope 31 is engaged with the stopper 38. By the engagement part between the engaging claw 36 and the stopper 38, the internal conduit 2 of the tube 7 is maintained in the closed state.

The following advantageous effects can be obtained by the above-described structure. Specifically, in the disposable endoscope 31 of the present embodiment, after use for, e.g. endoscopy, the push button 35 of the input section 32 is pushed to the pushed position shown in FIG. 7. Thereby, the tube 7 is collapsed by the push button 35, the deformation portion 37 is formed and the internal conduit 2 of the tube 7 is liquid-lightly closed. Hence, the liquid, such as a body liquid, which has flown in the tube 7, can be prevented from flowing out of the internal conduit 2 of the tube 7. As a result, the liquid, such as a body liquid, which stays in the internal conduit 2 of the tube 7 of the disposable endoscope 31, which has already been used, does not leak to the outside from the internal conduit 2 of the tube 7. Therefore, after the disposable endoscope 31 has been used for an infected patient, it is possible to reduce occasions where the source of infection spreads.

In addition, in the present embodiment, another push button 35 of the same structure may also be provided at the distal end portion of the tube body 8, and closing means of the tube 7 may be formed. In this case, since both end portions of the tube 7 of the disposable endoscope 31, which has already been used, can be closed. Therefore, advantageously, it becomes possible to more surely prevent the liquid, such as a body liquid, which stays in the internal conduit 2 of the tube 7, from leaking to the outside from the internal conduit 2 of the tube 7.

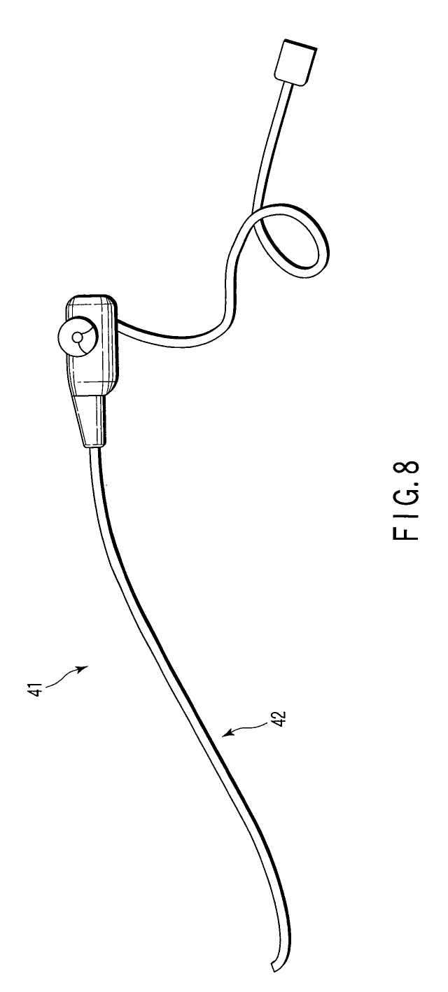

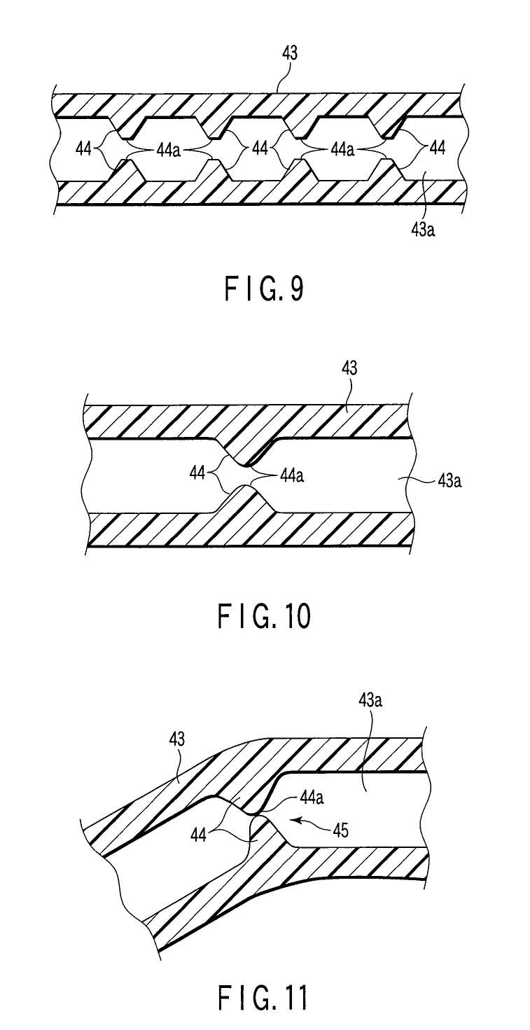

FIG. 8 to FIG. 12 show a fourth embodiment of the present invention. FIG. 8 shows the state in which a disposable endoscope 41 of the present embodiment is used. In this embodiment, a flexible tube 43 having the structure shown in FIG. 9 is provided as a channel tube, such as a therapeutic device insertion channel, in an insertion section 42 of the disposable endoscope 41.

As shown in FIG. 9, a plurality of inward projection portions 44 are juxtaposedly provided within the flexible tube 43 along the center axis of the flexible tube 43. The interval of every two inward projection portions 44, which neighbor in the back-and-forth direction of the flexible tube 43, is set at a proper value.

In the state in which the flexible tube 43 of the present embodiment is extended substantially straight, the respective inward projection portions 44 of the flexible tube 43 are held in the state in which distal end portions 44a are spaced apart, as shown in FIG. 10, by the resilient force of the flexible tube 43 itself. Thus, in this state, the flexible tube 43 is held in the open state.

In the case where the flexible tube 43 is bent over a predetermined diameter or more, the distal end portions 44a of the inward projection portions 44 of the flexible tube 43 come in contact with each other, as shown in FIG. 11. In this case, the internal conduit 43a of the flexible tube 43 is liquid-tightly closed. In the flexible tube 43 of the present embodiment, the contact parts between the distal end portions 44a of the inward projection portions 44 constitute closing means 45 which can liquid-tightly close the internal conduit 43a of the flexible tube 43.

Next, the operation of the above-described structure is described. At a time of normal use, the disposable endoscope 41 of the present embodiment is used in the state in which the insertion section 42 is extended substantially straight, as shown in FIG. 8. At this time, the respective inward projection portions 44 of the flexible tube 43 are held in the state in which the distal end portions 44a are spaced apart, as shown in FIG. 10. Thus, in this state, the flexible tube 43 is held in the open state.

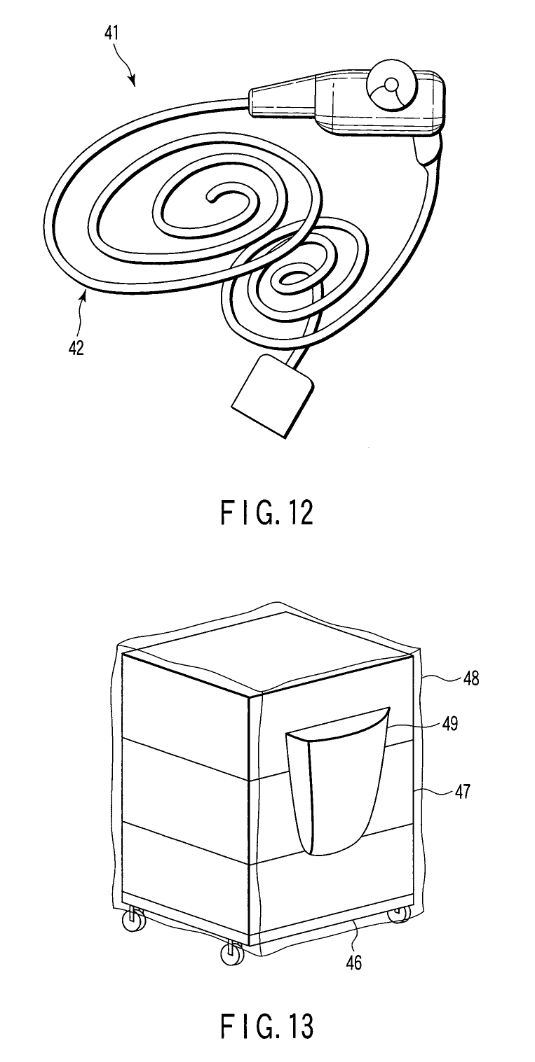

After use of the disposable endoscope 41, the entire insertion section 42 is wound with a relatively small diameter, as shown in FIG. 12. In this case, the distal end portions 44a of the inward projection portions 44 of the flexible tube 43 come in contact with each other, as shown in FIG. 11. In this case, the internal conduit 43a of the flexible tube 43 is liquid-tightly closed.

In addition, in the present embodiment, the disposable endoscope 41, which has already been used, is contained in a refuse bag (not shown) and discarded in the state in which the entire insertion section 42 is wound with a relatively small diameter, as shown in FIG. 12. Thus, maintaining means for maintaining the internal conduit 43a of the flexible tube 43 in the liquid-tightly closed state is constituted by container means such as the refuse bag.

The following advantageous effects can be obtained by the above-described structure. Specifically, in the disposable endoscope 41 of the present embodiment, after use for, e.g. endoscopy, the entire insertion section 42 is wound with a relatively small diameter, as shown in FIG. 12. Thereby, the distal end portions 44a of the inward projection portions 44 of the flexible tube 43 come in contact with each other, as shown in FIG. 11, and the internal conduit 43a of the flexible tube 43 is liquid-tightly closed. Thus, the liquid, such as a body liquid, which has flown in the flexible tube 43, can be prevented from flowing out of the internal conduit 43a of the flexible tube 43. As a result, the liquid, such as a body liquid, which stays in the internal conduit 43a of the flexible tube 43 of the disposable endoscope 41, which has already been used, does not leak to the outside from the internal conduit 43a of the flexible tube 43. Therefore, after the disposable endoscope 41 has been used for an infected patient, it is possible to reduce occasions where the source of infection spreads.

FIG. 13 shows the state in which a refuse bag 49 is attached to a drape 48 which is placed over a medical device 47 constituting an endoscope system mounted on a cart 46. The used disposable endoscope 41 in the state in which the entire insertion section 42 is wound with a relatively small diameter, as shown in FIG. 12, is contained, for example, in the refuse bag 49. The used disposable endoscope 41 in the refuse bag 49 is discarded in the state in which the disposable endoscope 41 is put together with the drape 48.

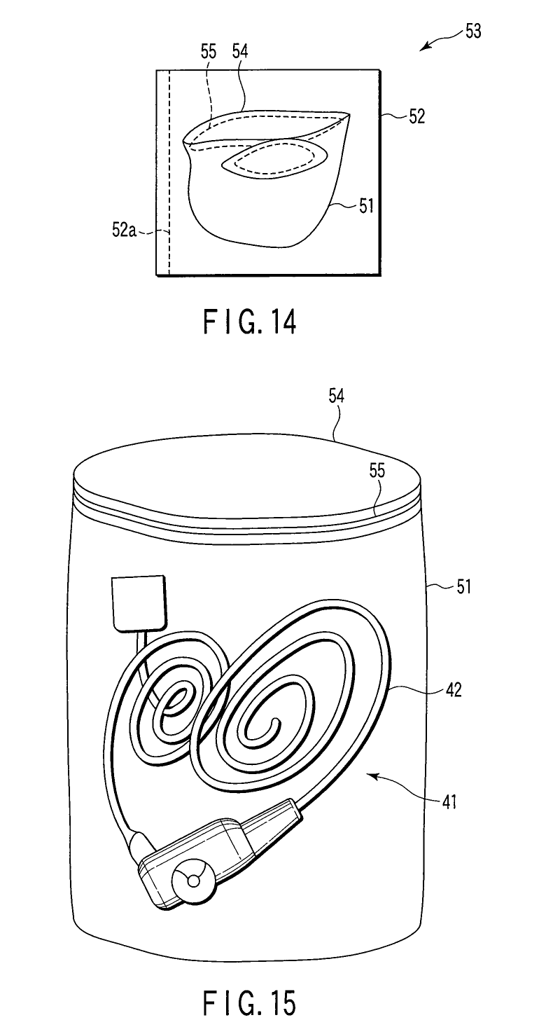

FIG. 14 shows an example of a refuse-bag-contained package 53 wherein a refuse bag 51 is put in a transparent bag 52. A ring-shaped resin rod 55 with resiliency is inserted in the entire peripheral part of an opening portion 54 of the refuse bag 51. The refuse bag 51 is stored in the refuse bag 52 in the state in which the resin rod 55 is twisted and folded compactly. A cutting line, such as perforation 52a, is formed on one end portion of the bag 52.

When the refuse bag 51 is to be used, the bag 52 is cut along the perforation 52a and the refuse bag 51 is taken out of the bag 52. At this time, the resin rod 55 is opened in a ring shape, and the opening portion 54 of the refuse bag 51 is broadened. Thus, the used disposable endoscope 41 can easily be stored in the refuse bag 51 from the opening portion 54 of the refuse bag 51.

In addition, in the state in which the used disposable endoscope 41 is stored in the refuse bag 51, the resin rod 55 is pulled out of the opening portion 54 of the refuse bag 51. Thereby, the opening portion 54 of the refuse bag can be easily fastened.

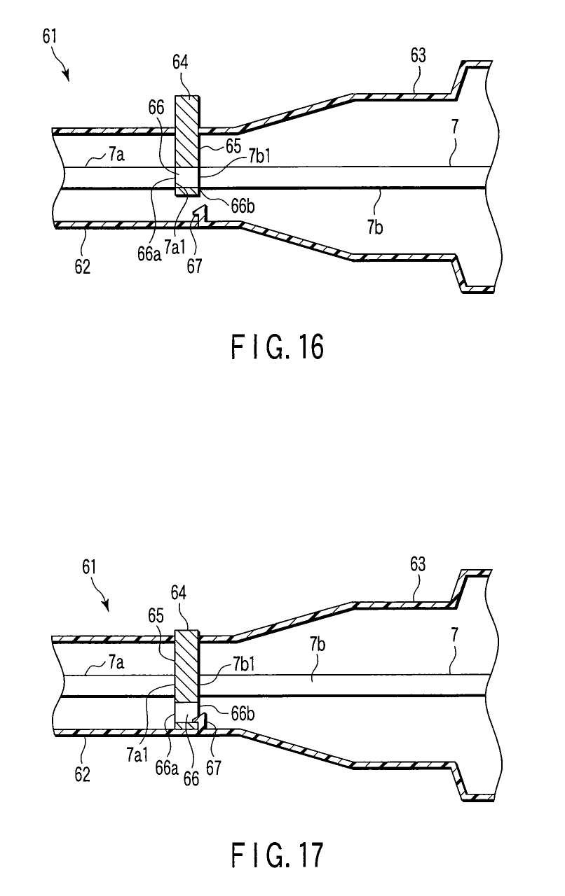

FIG. 16 and FIG. 17 show a fifth embodiment of the present invention. In the present embodiment, the structure of the input section 32 in the case of closing the tube 7 of the disposable endoscope 31 of the third embodiment (see FIG. 6 and FIG. 7) is altered as described below.

Specifically, in a disposable endoscope 61 of the present embodiment, a switch valve 64 for opening/closing the internal conduit 2 of the tube 7 is provided on a coupling part between an elongated insertion section 62 and a proximal-side operation section 63.

The switch valve 64 is provided on an intermediate portion of the tube 7. The switch valve 64 is attached so as to be slidable in a direction perpendicular to the direction of extension of the tube 7. The tube 7 is separated into a portion 7a on the distal-end side of the switch valve 64, and a portion 7b on the rear-end side of the switch valve 64. A rear end opening portion (first opening portion) 7a1 of the distal-end-side portion 7a and a distal end opening portion (second opening portion) 7b1 of the rear-end-side portion 7b are disposed to be opposed to the switch valve 64.

The switch valve 64 is supported to be movable from a non-operation position shown in FIG. 16 to an operation position shown in FIG. 17. Normally, the switch valve 64 is held in the non-operation position shown in FIG. 16 by holding means (not shown). At this time, one end portion of the switch valve 64 is held in a projecting position, as shown in FIG. 16, and projects to the outside of the insertion section 62.

Further, in the switch valve 64, a communication hole 66 is formed in a plate-shaped valve body (closing means) 65. The distal-end side of the communication hole 66 is provided with a third opening portion 66a which is communicable with the rear end opening portion 7a1 of the distal-end-side portion 7a of the tube 7, and the rear-end side of the communication hole 66 is provided with a fourth opening portion 66b which is communicable with the distal end opening portion 7b1 of the rear-end-side portion 7b of the tube 7. In the case where the switch valve 64 is held in the non-operation position shown in FIG. 16, the switch valve 64 is kept in the state in which the third opening portion 66a of the communication hole 66 communicates with the rear end opening portion 7a1 of the distal-end-side portion 7a of the tube 7, and the fourth opening portion 66b of the communication hole 66 communicates with the distal end opening portion 7b1 of the rear-end-side portion 7b.

A hook-shaped engaging claw 67 is provided on an inner wall surface on the proximal end side of the insertion section 62. In the case where the switch valve 64 is held in the non-operation position shown in FIG. 16, the engaging claw 67 is held in the state of disengagement from the switch valve 64.

In the case where the switch valve 64 has been moved to the operation position shown in FIG. 17, the engaging claw 67 is inserted and engaged in the communication hole 66 from the fourth opening portion 66b of the switch valve 64. At this time, the rear end opening portion 7a1 of the distal-end-side portion 7a of the tube 7 and the distal end opening portion 7b1 of the rear-end-side portion 7b of the tube 7 are closed by the valve body 65 of the switch valve 64. The engaging claw 67 is configured such that the engaging claw 67, if once engaged in the communication hole 66 from the fourth opening portion 66b of the switch valve 64, is disengageable.

Next, the operation of the above-described structure is described. At a time of normal use, the switch valve 64 is held in the non-operation position shown in FIG. 16. In this state, the tube 7 is held in the open state.

After use of the disposable endoscope 61, the switch valve 64 is operated and pushed in. At this time, in the case where the switch valve 64 has been pushed to the operation position shown in FIG. 17, the rear end opening portion 7a1 of the distal-end-side portion 7a of the tube 7 and the distal end opening portion 7b1 of the rear-end-side portion 7b of the tube 7 are closed by the valve body 65 of the switch valve 64. Thereby, the tube 7 is closed by the switch valve 64, and the internal conduit 2 of the tube 7 is liquid-tightly closed.

At the same time, the engaging claw 67 of the disposable endoscope 61 is engaged in the communication hole 66 from the fourth opening portion 66b of the switch valve 64. By the coupling part between the engaging claw 67 and communication hole 66, the switch valve 64 is held in the operation position shown in FIG. 17, and the internal conduit 2 of the tube 7 is maintained in the closed state.

The following advantageous effects can be obtained by the above-described structure. Specifically, in the disposable endoscope 61 of the present embodiment, after use for, e.g. endoscopy, the switch valve 64 is pushed to the operation position shown in FIG. 17. Thereby, the internal conduit 2 of the tube 7 is liquid-lightly closed. Thus, the liquid, such as a body liquid, which has flown in the tube 7, can be prevented from flowing out of the internal conduit 2 of the tube 7. As a result, the liquid, such as a body liquid, which stays in the internal conduit 2 of the tube 7 of the disposable endoscope 61, which has already been used, does not leak to the outside from the internal conduit 2 of the tube 7. Therefore, after the disposable endoscope 61 has been used for an infected patient, it is possible to reduce occasions where the source of infection spreads.

In addition, in the present embodiment, another switch valve 64 of the same structure may also be provided at the distal end portion of the insertion section 62, thereby to form the closing means of the tube 7. In this case, since both end portions of the tube 7 of the disposable endoscope 61, which has already been used, can be closed. Therefore, advantageously, it becomes possible to more surely prevent the liquid, such as a body liquid, which stays in the internal conduit 2 of the tube 7, from leaking to the outside from the internal conduit 2 of the tube 7.

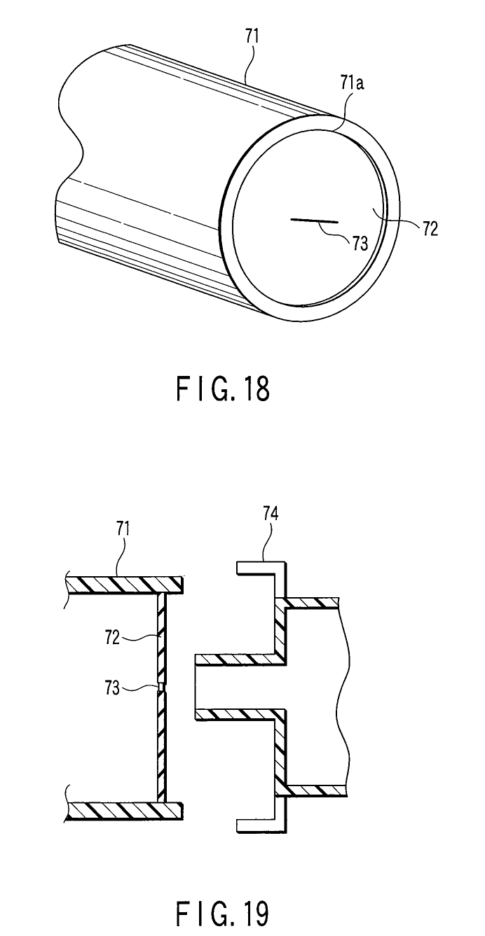

FIG. 18 and FIG. 19 show a sixth embodiment of the present invention. In this embodiment, a membrane-like closing member 72 serving as closing means, which is formed of an elastic material such as a rubber member, is provided on an opening end portion 71a of a flexible tube 71 of a channel tube such as a therapeutic device insertion channel, which is provided within the insertion section of the disposable endoscope. A slit 73, or a small hole, is formed at a substantially central part of the closing member 72. In the natural state, the slit 73, or a small hole, is held in the closed state by the elastic force of the elastic material of the closing member 72.

In the case where a connector 74 of an external device, for example, as shown in FIG. 19, is connected to the opening end portion 71a of the flexible tube 71, the closing member 72 is elastically deformed in a spreading fashion from the part of the slit 73. Thereby, the opening end portion 71a of the flexible tube 71 is opened, and the flexible tube 71 is connected to the connector 74 of the external device. In the case where the connector 74 of the external device is removed from the opening end portion 71a of the flexible tube 71, the closing member 72 of the opening end portion 71a of the flexible tube 71 elastically restores to the state of closing the opening end portion 71a of the flexible tube 71.

The following advantageous effects can be obtained by the above-described structure. Specifically, in the case where the connector 74 of the external device, for instance, is removed after use from the opening end portion 71a of the flexible tube 71 in the present embodiment, the closing member 72 elastically restores to the state of closing the opening end portion 71a of the flexible tube 71. Thereby, in the case where the connector 74 of the external device, for instance, is removed from the opening end portion 71a of the flexible tube 71, the opening end portion 71a of the flexible tube 71 can automatically be closed by the closing member 72. Thus, the liquid, such as a body liquid, which has flown in the flexible tube 71, can be prevented from flowing out of the internal conduit of the flexible tube 71. As a result, the liquid, such as a body liquid, which stays in the flexible tube 71 of the disposable endoscope, which has already been used, does not leak to the outside from the inside of the flexible tube 71. Therefore, after the disposable endoscope has been used for an infected patient, it is possible to reduce occasions where the source of infection spreads.

FIG. 20 to FIG. 22 show a seventh embodiment of the present invention. In the present embodiment, the structure of the closing member 72 of the flexible tube 71 of the sixth embodiment (see FIG. 18 and FIG. 19) is altered as described below.

Specifically, in the present embodiment, a closing member 81, which is formed of an elastic member, such as a rubber plate, in a substantially conical shape, as shown in FIG. 22, is provided on the opening end portion 71a of the flexible tube 71. An annular fixed portion 81a, which is attached and fitted over the opening end portion 71a of the flexible tube 71, is formed at a proximal end portion of the closing member 81 of the present embodiment. A taper portion 81b is formed at a distal end portion of the closing member 81. A split portion 81c, such as a notch or a slit, is formed at the distal end of the taper portion 81b.

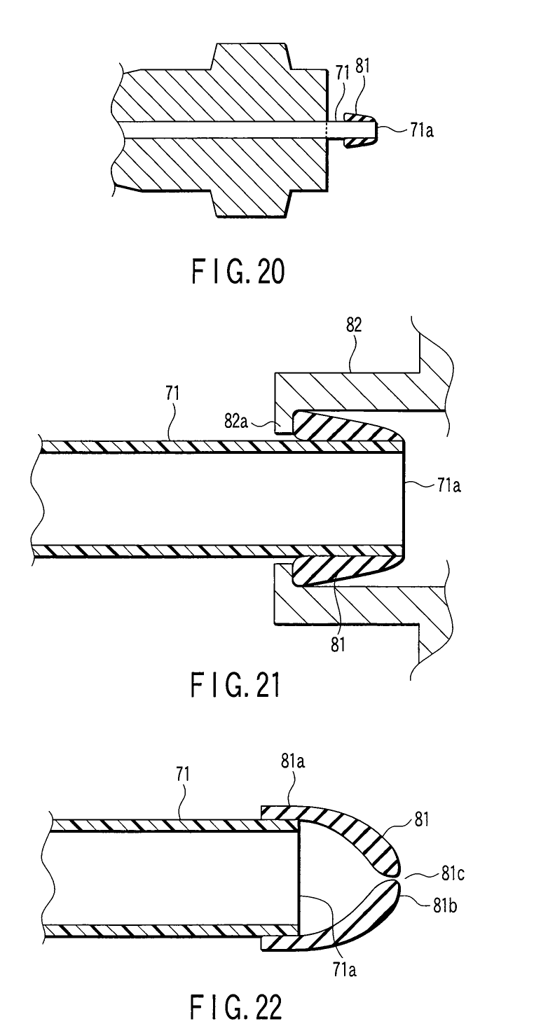

The closing member 81 of the present embodiment is set on the opening end portion 71a of the flexible tube 71 in the state in which the split portion 81c of the taper portion 81b is elastically deformed in a reversed state. Besides, as shown in FIG. 21, a claw portion 82a, which projects inward, is provided at a distal end portion of a connector 82 of an external device, which is used in the present embodiment.

In the case where the connector 82 of the external device is removed from the opening end portion 71a of the flexible tube 71, the closing member 81 is pulled back by the claw portion 82a at the time of the operation of removing the connector 82. Thereby, as shown in FIG. 22, the closing member 81 of the opening end portion 71a of the flexible tube 71 elastically restores to the state of closing the opening end portion 71a of the flexible tube 71.

The following advantageous effects can be obtained by the above-described structure. Specifically, in the case where the connector 82 of the external device, for instance, is removed after use from the opening end portion 71a of the flexible tube 71 in the present embodiment, the closing member 81 elastically restores to the state of closing the opening end portion 71a of the flexible tube 71. Thereby, in the case where the connector 82 of the external device, for instance, is removed from the opening end portion 71a of the flexible tube 71, the opening end portion 71a of the flexible tube 71 can automatically be closed by the closing member 81. Thus, the liquid, such as a body liquid, which has flown in the flexible tube 71, can be prevented from flowing out of the internal conduit of the flexible tube 71. As a result, the liquid, such as a body liquid, which stays in the flexible tube 71 of the disposable endoscope, which has already been used, does not leak to the outside from the inside of the flexible tube 71. Therefore, after the disposable endoscope has been used for an infected patient, it is possible to reduce occasions where the source of infection spreads.

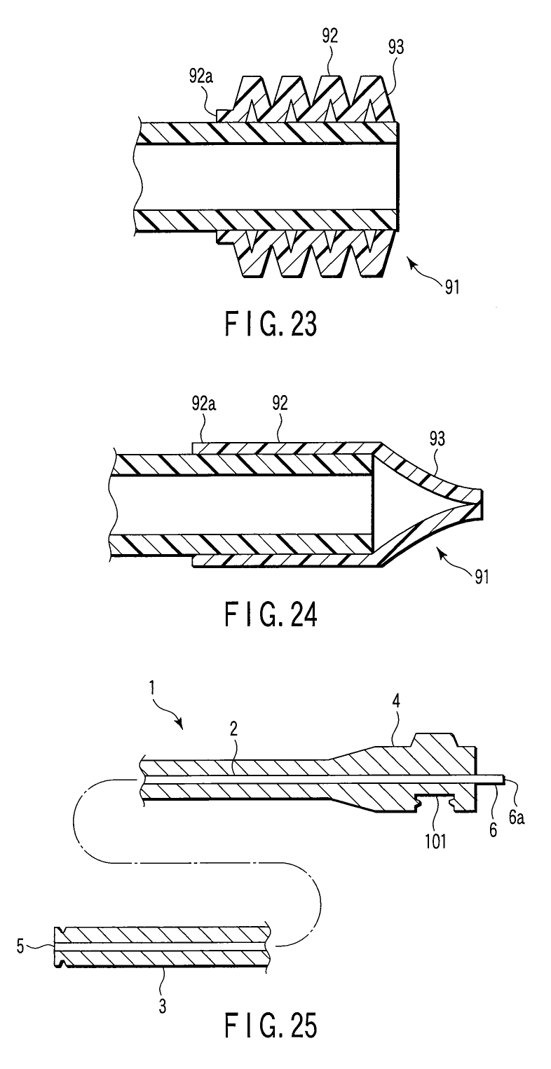

FIG. 23 and FIG. 24 show an eighth embodiment of the present invention. In the present embodiment, the structure of the closing member 81 of the seventh embodiment (see FIG. 20 to FIG. 22) is altered as described below.

Specifically, in a closing member 91 in the present embodiment, a narrowed portion 93, which is formed in a substantially conical shape, as shown in FIG. 24, is provided at a distal end portion of an elastic cylindrical member 92. An annular fixed portion 92a, which is fixed on an outer peripheral surface of the flexible tube 71, is formed at a proximal end portion of the cylindrical member 92

The closing member 91 of the present embodiment, as shown in FIG. 23, is set on the flexible tube 71 in the state in which the narrowed portion 93 is elastically deformed in a bellows-like folded state.

In the case where the connector 82 (see FIG. 21) of the external device is removed from the opening end portion 71a of the flexible tube 71, the narrowed portion 93, which is folded in the bellows-like fashion, is pulled back by the claw portion 82a at the time of the operation of removing the connector 82. Thereby, as shown in FIG. 24, the closing member 91 of the opening end portion 71a of the flexible tube 71 elastically restores to the state in which the opening end portion 71a of the flexible tube 71 is closed by the narrowed portion 93.

The following advantageous effects can be obtained by the above-described structure. Specifically, in the case where the connector 82 of the external device, for instance, is removed after use from the opening end portion 71a of the flexible tube 71 in the present embodiment, the narrowed portion 93 of the closing member 91 elastically restores to the state of closing the opening end portion 71a of the flexible tube 71. Thereby, in the case where the connector 82 of the external device, for instance, is removed from the opening end portion 71a of the flexible tube 71, the opening end portion 71a of the flexible tube 71 can automatically be closed by the closing member 91. Thus, the liquid, such as a body liquid, which has flown in the flexible tube 71, can be prevented from flowing out of the internal conduit of the flexible tube 71. As a result, the liquid, such as a body liquid, which stays in the flexible tube 71 of the disposable endoscope, which has already been used, does not leak to the outside from the inside of the flexible tube 71. Therefore, after the disposable endoscope has been used for an infected patient, it is possible to reduce occasions where the source of infection spreads.

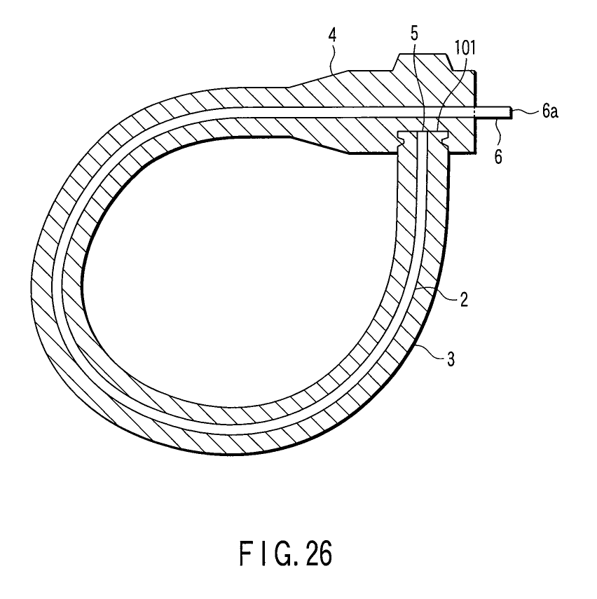

FIG. 25 and FIG. 26 show a ninth embodiment of the present invention. In the present embodiment, a recess-shaped engaging portion 101 is provided in an outer peripheral surface of the operation section 4 of the disposable endoscope 1 of the first embodiment (see FIG. 1 to FIG. 3). This engaging portion 101 can be engaged with a distal end portion of the insertion section 3.

At a time of normal use, the disposable endoscope 1 of the present embodiment is held in a fixed position shown in FIG. 25. In this state, the distal-end opening portion 5 at the distal end of the insertion section 3 and the opening end 6a of the coupling end portion 6 at the rear end of the conduit 2 are kept open.

After use of the disposable endoscope 1, as shown in FIG. 26, the distal end portion of the insertion section 3 is fitted and engaged in the engaging portion 101 of the operation section 4. Thereby, the internal conduit 2 of the disposable endoscope 1 is liquid-tightly closed.

The following advantageous effects can be obtained by the above-described structure. Specifically, after use of the disposable endoscope 1 of the present embodiment, the distal end portion of the insertion section 3 is fitted and engaged in the engaging portion 101 of the operation section 4. Thus, the liquid, such as a body liquid, which has flown in the internal conduit 2 of the disposable endoscope 1, can be prevented from flowing out of the internal conduit 2 of the disposable endoscope 1. As a result, the liquid, such as a body liquid, which stays in the internal conduit 2 of the disposable endoscope 1, which has already been used, does not leak to the outside from the internal conduit 2 of the disposable endoscope 1. Therefore, after the disposable endoscope 1 has been used for an infected patient, it is possible to reduce occasions where the source of infection spreads.

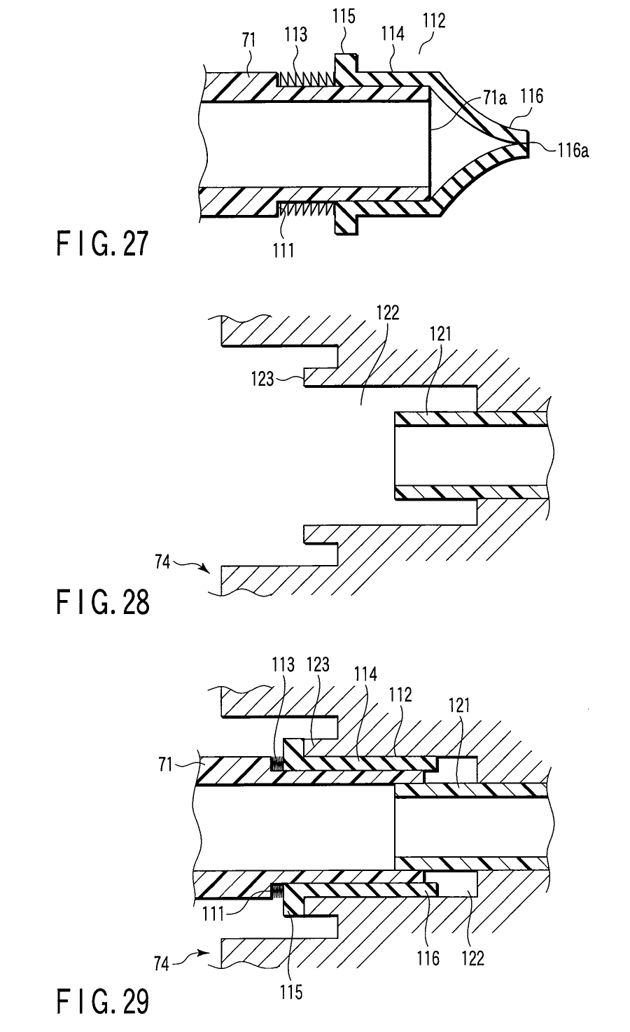

FIG. 27 to FIG. 29 show a tenth embodiment of the present invention. In the present embodiment, the structure of the closing member 72 of the flexible tube 71 of the sixth embodiment (see FIG. 18 and FIG. 19) is altered as described below.

Specifically, in the present embodiment, an annular recess portion 111 is provided on an outer peripheral portion in the vicinity of the opening end portion 71a of the flexible tube 71. The recess portion 111 is provided with a cylindrical closing member 112 and an urging member 113 which is formed of a coil spring. The closing member 112 is provided along the recess portion 111 of the flexible tube 71 so as to be movable in the axial direction of the flexible tube 71. The urging member 113 urges the closing member 112 toward the opening end portion 71a of the flexible tube 71.

The closing member 112 includes a cylindrical body 114 which is formed of an elastic material such as rubber. An outwardly projecting flange portion 115 is formed at a proximal end portion of the cylindrical body 114. In addition, as shown in FIG. 27, a closing portion 116 having a substantially conical, tapered distal end portion is formed at a distal end portion of the cylindrical portion 114. A joining portion 116a, which is joined in an openably/closably closed state, is provided at a central part of the closing portion 116.

In the natural state, as shown in FIG. 27, the closing portion 116 of the cylindrical body 114 of the closing member 112 is projected outward from the opening end portion 71a of the flexible tube 71 by the urging force of the urging member 113, and the joining portion 116a of the closing portion 116 is held in the joined, closed state. At this time, the opening end portion 71a of the flexible tube 71 is held in the closed state by the closing portion 116 of the closing member 112.

The connector 74 of the external device is provided with a coupling conduit 121 which is inserted, when connected to the flexible tube 71, in the opening end portion 71a of the flexible tube 71 and is connected to the flexible tube 71. A circular coupling hole portion 122, which has a greater diameter than the coupling conduit 121, is formed around the coupling conduit 121. A circular-cylindrical pressing ring 123 is formed at a distal end portion of the coupling hole portion 122. The outside diameter of the coupling conduit 121 is set to be slightly less than the inside diameter of the flexible tube 71. The inside diameter of the coupling hole portion 122 is set to be slightly greater than the outside diameter of the cylindrical body 114.

At a time of a connecting work of connecting the connector 74 of the external device, as shown in FIG. 28, to the opening end portion 71a of the flexible tube 71, the closing member 112 side of the flexible tube 71 is inserted in the coupling hole portion 122 of the connector 74 of the external device. At this time, as shown in FIG. 27, the closing member 112 of the flexible tube 71 is held in the closed state in which the joining portion 116a of the closing portion 116 is joined. In this state, the closing member 112 side of the flexible tube 71 is pushed into the coupling hole portion 122 of the connector 74 of the external device. Then, at first, the pressing ring 123 comes in contact with the flange portion 115 of the cylindrical body 114. If the closing member 112 side of the flexible tube 71 is further pushed, the closing member 112 is moved by the pressing force of the pressing ring 123 to the left in FIG. 27 against the urging force of the urging member 113. By the movement of the closing member 112, the joining portion 116a of the closing portion 116 of the closing member 112 is elastically deformed in a direction of opening. Thus, as shown in FIG. 29, in the state in which the opening end portion 71a of the flexible tube 71 is opened, the coupling conduit 121 is inserted in the opening end portion 71a of the flexible tube 71 and is connected to the flexible tube 71. Thereby, the opening end portion 71a of the flexible tube 71 is opened and connected to the connector 74 of the external device, for instance.

In the case where the connector 74 of the external device, for instance, is removed from the opening end portion 71a of the flexible tube 71, the closing member 112 is pressed toward the opening end portion 71a of the flexible tube 71 by the urging force of the urging member 113. Accordingly, the closing member 112 of the flexible tube 71 is pushed out in such a direction as to project outward of the opening end portion 71a of the flexible tube 71. Thereby, as shown in FIG. 27, the joining portion 116a of the closing portion 116 elastically restores to the joined, closed state. Thus, the closing member 112 of the opening end portion 71a of the flexible tube 71 is automatically switched to the state of closing the opening end portion 71a of the flexible tube 71.

The following advantageous effects can be obtained by the above-described structure. Specifically, in the case where the connector 74 of the external device, for instance, is removed after use from the opening end portion 71a of the flexible tube 71 in the present embodiment, the closing member 112 is pushed toward the opening end portion 71a of the flexible tube 71 by the urging force of the urging member 113, and the closing member 112 elastically restores to the state of closing the opening end portion 71a of the flexible tube 71. Thereby, when the connector 74 of the external device, for instance, is removed from the opening end portion 71a of the flexible tube 71, the opening end portion 71a of the flexible tube 71 can automatically be closed by the closing member 112. Thus, the liquid, such as a body liquid, which has flown in the flexible tube 71, can be prevented from flowing out of the internal conduit of the flexible tube 71. As a result, the liquid, such as a body liquid, which stays in the flexible tube 71 of the disposable endoscope, which has already been used, does not leak to the outside from the inside of the flexible tube 71. Therefore, after the disposable endoscope has been used for an infected patient, it is possible to reduce occasions where the source of infection spreads.

The present invention is not limited to the above-described embodiments. Needless to say, various modifications may be made without departing from the spirit of the invention.

The invention is effective in the technical field of manufacturing and using a medical device, medical equipment and an endoscope for disposable use, which are, if once used, discarded without being reused.

What is claimed is:

1. A medical device comprising:
a communication member including a first opening portion, a second opening portion, and a communication conduit for communication between the first opening portion and the second opening portion;
a closing portion capable of liquid-tightly closing the communication conduit; and
a maintaining member capable of maintaining the closing portion in a closed state.

2. The medical device according to claim 1, further comprising:
a deformation portion provided on the communication conduit as the closing portion; and
an input section which deforms the deformation portion.

3. The medical device according to claim 2, further comprising:
a first holding portion and a second holding portion which are provided on both sides of the deformation portion on the communication conduit; and
a rotation section enabling relative rotation between the first holding portion and the second holding portion, with the deformation portion being interposed.

4. The medical device according to claim 2, further comprising:
a medical device body through which the communication conduit is passed; and
an operation section which operates the input section from outside of the medical device body.

5. The medical device according to claim 2, wherein:
the deformation portion is closed when the medical device is bent over a predetermined diameter or more; and
the maintaining member maintains the state in which the medical device is bent over the predetermined diameter or more.

6. The medical device according to claim 1, including:
a third opening portion and a fourth opening portion which are formed, with a gap portion interposed, between the first opening portion and the second opening portion which are provided on the communication conduit;
the closing portion which is slidably provided at the gap portion and includes an opening window which permits communication between the third opening portion and the fourth opening portion, and a closing portion which enables closing between the third opening portion and the fourth opening portion; and
the maintaining member capable of fixing the closing member in a state in which the third opening portion and the fourth opening portion are closed by the closing portion.

7. The medical device according to claim 1, further comprising:
an attachment portion which liquid-tightly attaches the closing portion to the first opening portion or the second opening portion; and
an urging member for urging the closing portion in a closed state.

8. The medical device according to claim 1, wherein:
the maintaining member includes an engaging portion provided on an outer wall of the medical device, the engaging portion being capable of engaging the communication conduit, the first opening portion or the second opening portion; and
the closing member includes a sealing portion provided on an outer wall of the medical device, the sealing portion being capable of sealing the first opening portion or the second opening portion.

9. Medical equipment comprising:
the medical device of claim 1;
an external device to which the medical device is connected; and
a communication maintaining member, provided on the external device, for preventing the closing portion from closing the communication conduit while the medical device is being used.

10. An endoscope including the medical device of claim 1, the endoscope including at least one of an insertion section and a universal cord,
the closing portion being provided at least one of the insertion section and the universal cord.
